(19) United States
(12) Patent Application Publication
(10) Pub. No.: US 2011/0184320 A1
(43) Pub. Date: Jul. 28, 2011

(54) MEASUREMENT SYSTEM USING BODY MOUNTED PHYSICALLY DECOUPLED SENSOR

(76) Inventors: J. Clay Shipps, Baltimore, MD (US); Hikmet Andic, Baltimore, MD (US)

(21) Appl. No.: 13/014,014

(22) Filed: Jan. 26, 2011

Related U.S. Application Data
(60) Provisional application No. 61/298,388, filed on Jan. 26, 2010.

(51) Int. Cl. A61B 5/11

(52) U.S. Cl. 600/595

(57) ABSTRACT

A system for monitoring a user's body has a single sensor patch directly affixed to the user's body. The sensor senses a condition of the user's body and transmits the sensed signal directly to a base station located remote from the sensor. The sensor can alternatively transmit the sensed signal to a receiver located in the user's helmet. The helmet optionally includes a power module which wirelessly provides power to the sensor. Since the sensor is affixed to the user, and not the helmet, it will only record impact to the user's body. Once the helmet is removed from the user's head, the sensor is powered off or can remain charged for a short period of time by a chargeable power source to record any subsequent impact to the user's body.

### RELATED APPLICATIONS

This application claims the benefit of U.S. Provisional Application No. 61/298,388, filed Jan. 26, 2010, the entire contents of which are incorporated herein by reference.

### BACKGROUND OF THE INVENTION

1. Field of Invention

The present invention is directed to a single sensor patch to be directly mounted to the body of a user and directly communicating with a remotely located base station. The invention can also include a proximity device located in a proximity garment for providing power to the sensor patch and recording, storing and transmitting data from the sensor while being in close proximity to, but physically decoupled from, the sensor.

2. Background of the Invention

Individuals in contact and high speed sports are often exposed to impacts to their head that can be damaging to the brain. Individuals in the military are similarly exposed to impact events. History has shown that many individuals that have been exposed to repeated impact events over an extended period of time, or have been exposed to a very large impact event, tend to develop symptoms well after the event or as they grow older. In many cases it is suspected that the individual that begins to show symptoms of diminished physical and mental capabilities has had many concussions that have gone noticed and untreated. This is highly likely in many sporting events where multiple players are engaged simultaneously and head impacts occur on virtually every play. In most cases, the player must alert the medical staff or team personnel to the fact that they have experienced a significant impact. Utilizing the player as an alert mechanism is not a reliable method of determining an impact on a player because the player may be motivated by other factors and down play the impact event. In the case of military personnel, they may have been exposed to a head impact event but due to the stress of the situation and the proximity to medical personnel may delay reporting the event or be delayed in receiving treatment.

If it is suspected that an individual has been exposed to an impact, a physician can conduct cognitive testing on the individual and determine if their brain function is “normal” or, if available, can compare the results of this testing to a baseline acquired on the individual at an earlier date. A physician can also generate images of the brain to determine if the brain has been damaged and to locate the damaged area. However, in all head injury cases in the sports and military, the treating physician has no quantifiable information on the impact to the individual's head.

To date, there have been no definitive, quantitative studies on humans that show how specific impacts to a person's head affect their mental or physical capabilities in the short term or long term. For instance, it is not well known if five impacts of 10 g to a specific location of an individual's head over a specific amount of time have the same affect as one 50 g impact in the same location or two 25 g impacts over the same amount of time. Using a boxing analogy, it is not well known if the 60 jabs in a fight or the 5 large blows to the side of the head, or a combination of those, will affect the fighter during the fight or long term after retirement. To begin to understand how the level, location, frequency, and accumulation of impact events affect an individual over time an accurate impact history needs to be acquired and monitored over a long period of time. In the short term, this impact history can be used to treat individuals quickly and can be used along with other testing to determine their ability to perform. In the long term, this history can be correlated with symptoms of diminished physical and mental capabilities and help to find ways to improve treatment for individuals and to improve protective gear worn by individuals.

Prior art systems have been developed in an attempt to measure impacts to an individual or sports players head. For instance, U.S. Patent Pub. No. 2009/0000377 (which corresponds to WO/2009/006406) entitled “Brain Impact Measurement System” is for a system comprised of an impact collection and registration system mounted to an individual's body and a hand held reader that takes the data and stores and displays it visually. The sensors are mounted in an ear piece that has to be worn by and individual. While some earpiece mounted sensors that locate the sensors deep in the wearer's ear canal have shown that they can accurately record head impacts, the individual's normal method of hearing is impacted and may affect their performance in a sporting event or in a military exercise. The content of U.S. Patent Pub. Nos. 2009/0000377 and WO/2009/006406 are incorporated by reference herein. However, these systems employ a helmet and place the measurement sensors within the helmet. The sensors were located in the helmet such that they come in contact with the outer surface of the players head when the helmet is placed on the player. The helmet, while often secured with a chin strap, can still move or slip about the head linearly and angularly during an impact. In some cases the helmet can be knocked off a player during an impact event. This slippage of the helmet about the head results in measurement error for both the linear and the angular acceleration measurements which can result in an inaccurate account of the impact level and location. The attachment of the sensors directly to the head using an adhesive patch eliminates interference with the wearer's hearing and also eliminates inaccuracies due to the helmet moving or slipping about the players head and can therefore provide a more accurate impact level and location.

### SUMMARY OF THE INVENTION

The present invention is directed to a single sensor patch to be mounted directly on the body of a user that has the capability of having a wireless interface that can transmit data wirelessly, be interrogated wirelessly and optionally be in proximity to a source of power, interrogation and transmission while being physically decoupled from the same. It is contemplated that the sensor can be used in various applications including but not limited to sports, military, medical, aeronautical, astronomical, firefighting, and scuba diving applications, etc. While normally to be mounted on the body of a human being, the sensor can also be mounted on the body of an animal.

The sensor can be used in a system that includes the sensor itself and a proximity interface that provides power, data acquisition, data storage and data transmission. The proximity interface does not have to be physically connected to the sensor, but can communicate with and power the sensor by merely being in sufficiently close proximity to the sensor. The sensor itself may have its own power source such as a battery and can be mountable to a user by being included in a mounting device such as a patch.

The present invention is illustrated by example in an embodiment directed to a system/device which characterizes the impact to an individual's head. The individual could be a sports person that wears a helmet or headgear in football, hockey, racing, lacrosse, skiing, boxing, etc., or a person in the military. For the purposes of explanation and example, a football player/helmet is being used, but it is to be understood that the invention is not limited to football or sports.

In the present invention, the sensors that measure the impact to the player's head are mounted directly onto the player's head using thin, possibly disposable, adhesive patches. The sensor patches can be standardized so that they are the same for all players. The other component which is optionally provided is a helmet or headgear. In professional sports, and possibly in the military, each individual has his/her own custom fitted helmet which is unique to the individual wearer.

The standardized sensor patches are placed directly on the player's head in specified locations. The sensor patches include a small battery to power them and a wireless transceiver to transmit and receive data. Alternatively or in addition, the sensor patches may be powered wirelessly by the helmet and are therefore only enabled when the player's helmet is on his head. Likewise, the helmet may only be powered and operational when it is located on the player's head.

As soon as the player removes his helmet, the recording of the time stamped impact information will cease. Therefore, the two components, the sensor patches and the helmet, must be used together to complete a circuit which allows the system to be powered, to collect impact data, to store that head impact information, and then transmit the desired impact information to a medical staff at a remote location. The amount and frequency of data transmission to the medical staff can be determined based on agreed upon guidelines for the organization in which the player is a member.

These and other objects of the invention, as well as many of the intended advantages thereof, will become more readily apparent when reference is made to the following description, taken in conjunction with the accompanying drawings.

### BRIEF DESCRIPTION OF THE DRAWINGS

FIG. 1 is a block diagram of the system electronics in accordance with a preferred embodiment of the invention having a single patch which communicates with a base station;

FIG. 2 shows positions for the sensor patch(es) to be placed on the head of a player;

FIG. 3 is a block diagram of an alternative embodiment of the invention having a repeater which is positioned in a helmet and communicates with the patch and the base station;

FIG. 4 is a block diagram of another alternative embodiment of the invention of FIG. 3, where the repeater provides power to the patch;

FIG. 5 shows front and side views of the repeater integrated in a helmet;

FIG. 6 is a block diagram of three identical patches provided on a single wearer and communicating with a base station, in accordance with an another embodiment of the invention; and

FIG. 7 is a block diagram of three different types of patches provided on a single wearer and communicating with a repeater which powers the patches, in accordance with another embodiment of the invention.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in similar manner to accomplish a similar purpose.

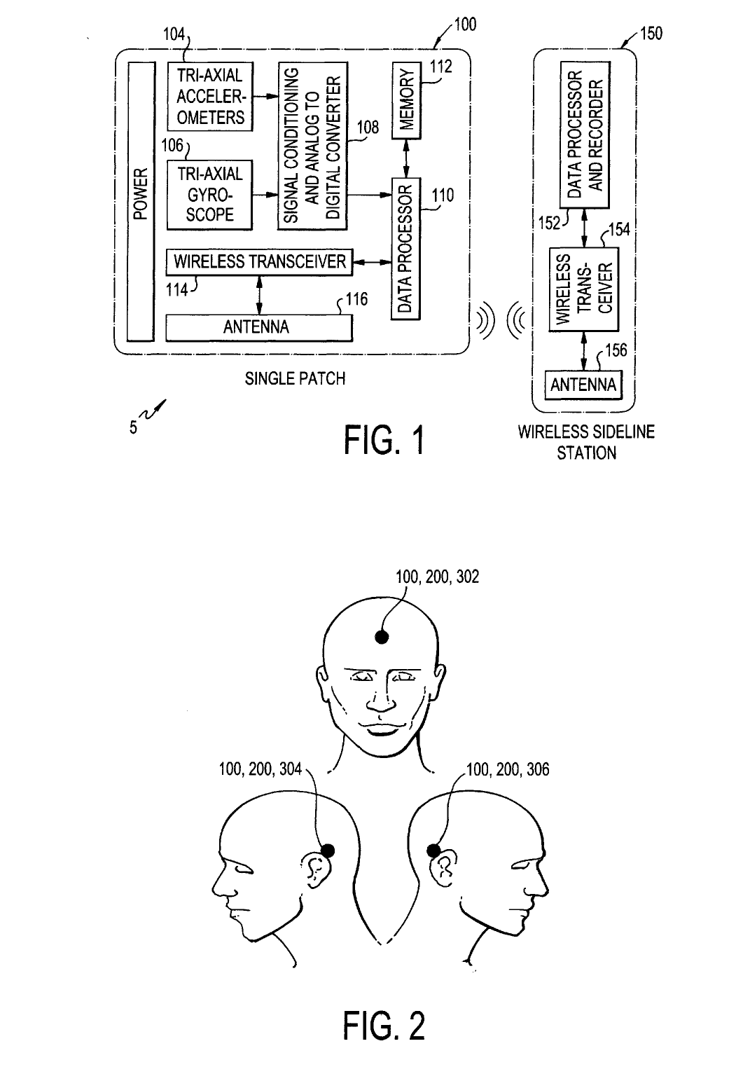

Turning to the drawings, FIG. 1 shows a head impact measurement system 5 having a single patch 100 for each individual, and a base station 150. The patch 100 carries power (battery) 102, tri-axial accelerometer sensors 104, tri-axial gyroscope sensors 106, signal conditioning and analog to digital converters (ADC) 108, data processor 100, memory bank or storage device 112, wireless transceiver 114 and an antenna 116. Each patch 100 has three individual accelerometers mounted orthogonally and three individual gyroscopes mounted orthogonally. Alternatively a single tri-axial accelerometer 104 can be used in addition to a single tri-axial gyroscope 106. In either configuration the combined tri-axial accelerometers and the tri-axial gyroscope will provide a 6 degree of freedom system which will completely characterize the movement of the wearers head. The sensor may be MEMS-based, mini-triax or other similar passive or active device and is not limited to the use of a processor 110, sensors 104, 106, ADC 108, memory 112, transceiver 114, and antenna 116.

The single patch 100 is mounted directly to an individual's body (e.g., the user's head). The patch 100 acquires impact data, processes that impact data, and sends it wirelessly to the sideline base station 150. The patch 100 can be programmed with thresholds to reduce the level or amount of data that is transmitted to the sideline station 150. The data thresholds will be preprogrammed in the patch 100, but can also be changed by the medical or support staff using the sideline wireless base station 150. In this embodiment, the patch 100 is always powered ON once placed on the individual's head. Therefore low power components are most desirable for use on the patch 100. To accommodate this, low power sensors 104, 106 can be MEMS devices or piezzo-electric sensors consuming micro to milliwatts, electronics 108, 110, 114, and wireless 114, 116 can be low power and utilize low power wireless protocol such as Bluetooth or Zigbee. These protocols are specifically designed toward portable, audio consumer electronics such as gaming, or phone earpieces. However, a proprietary wireless protocol could be used as well.

The patch 100 can be placed at any location on the individual's head, such as at any of the positions shown in FIG. 2. However, the location of the patch must be known relative to a reference coordinate system for the head. If the tri-axial accelerometers 104 do not measure down to DC, i.e. the gravitational component of acceleration, then the orientation of the patch relative to a chosen coordinate axis for the head is needed. In the preferred embodiment, the tri-axial accelerometer 104 measures the DC component of acceleration on all three axes and from that the orientation of the patch relative to the reference coordinate system for the head can be determined. The patch 100 is preferably an adhesive patch made of a flex circuit comprised of a flexible material such as kapton or paper, and/or the electronics can be mounted to a flexible substrate such as kapton or paper. The material may also be semi-flexible or semi-rigid as long as the patch on which the sensing elements 104, 106 are mounted is directly in contact with the user's body. The patch adhesive can be any suitable adhesive, such as used in the medical industry. The patch 100 is approximately one inch in diameter, though other suitable shapes and sizes can be utilized.

The base station 150 includes a processor 152, wireless transceiver 154, and an antenna 156. The processor 152 can have internal memory or be in communication with a separate memory or other storage device. The data processor 152 can also have a user input device, such as a mouse, touch display, and keyboard. The processor 152 can also be in communication with one or more output devices such as a display, audio system, and printer, to output the condition of the player. The outputs can include impact levels, locations, frequency, etc. as well as heart rate, brain function, temperature, or a report which analyzes that information. The base station 150 is located remotely from the patch 100. For instance, the patch 100 is typically located on a player playing on a playing field, and the base station 150 is located at the sideline of the playing field during play.

In operation, the patch 100 is synchronized with the base station 150 prior to placing the patch 100 on the player's body. For instance, the patch memory 112 can store a unique ID for the patch 100. The base station 150 can send an interrogation signal to the patch 100, which responds by indicating it's ID. That ID can then be stored in the base station processor 152 memory and associated with the player who will be wearing the patch 100, as well as the position on the player's head where the patch 100 is to be placed, and the orientation of the patch. Alternatively, the processor 152 can use a predefined ID, predefined head position, and/or predefined orientation for a particular player. That ID can be programmed into the patch memory 112 prior to placing the patch in the player's body, by transmitting the ID from the base station 150 via transceiver 154 and antenna 156, to be received by the patch 100 via transceiver 114 and antenna 116. The system includes tolerance to account for the small variations in head sizes and patch location. Alternatively, facial recognition system software can be used to accurately measure the location and/or orientation of a patch on the players' heads. Such systems take a picture of the player's head wearing the patch and detect the exact location of the patch using location recognition software. That would reduce any error associated with the varying patch location on the players' head. Patches 100 can be provided to multiple players and the base station 150 is in communication with those multiple patches 100; though each player only wears a single patch 100.

Once the patch 100 is turned on and placed on the players head, the patch is synchronized with the base station 150. The sensors 104, 106 sense the condition of the player's head (or body) and send that analog sensed signal to the signal conditioning and A/D converter 108, which processes the signal and generates a digital sensed signal. That digital sensed signal is then provided to the data processor 110. The processor 110 analyzes the signal. If the signal exceeds a certain threshold level or otherwise is a cause of concern, an alarm signal is generated. The signal and/or alarm conditions are stored in the memory 110, and are also immediately transmitted in real-time by the wireless transceiver 114 and the antenna 116 to the base station 150. The signal can be transmitted, for instance, by radio frequency, Bluetooth or Zigbee or any other protocols.

The base station 150 receives the transmitted digital sensed signal and/or alarm signal via the transceiver 154 and the antenna 156. The digital sensed signal and/or the alarm signal is then provided to the processor 152, where it is stored. The processor 152 can then analyze the data and provide results to the staff located at the sideline over one of the output devices. The processor 152 can also cause the information to be retransmitted to another remote processor, such as at a viewing booth or medical location. Accordingly, from the moment the sensed signal is sensed by the sensors 104, 106, base station 150 receives and analyzes the impact information in real-time during play. Though the sensed signal and alarm signal can be transmitted in real time, a power saving mode can choose to only transmit the alarm conditions during play to conserve power 102, and a full download of the patch memory 112 can be conducted when the player returns to the sideline, or after the patch 100 is removed. Or, the patch 100 need not do any analysis of the sensed signal, but instead only transmits the sensed signal; and the base station processor 152 can determine if there is an alarm condition to be generated.

The base station processor 152 analyzes the received sensed signal from the user wearing the sensor patch 100. That information can be used, for instance, by a medical team on the sideline of a football game, to determine whether a player has sustained an injury, should otherwise be removed from the game, or undergo an analysis.

The power supply 102 is a high energy density battery and voltage regulation circuitry. The battery can be a button size lithium ion as used in hearing aids, or can be flexible and an intrinsic part of the patch structure, i.e. it could be the patch material itself and carry the sensor and electronics. The tri-axial accelerometers 104 are preferably a single chip orthogonal tri-axial accelerometer or three uni-axial accelerometers mounted orthogonally. The tri-axial gyroscopes 106 are a single chip orthogonal tri-axial gyroscope or three uni-axial gyroscopes mounted orthogonally. The processor 110 is a low power data processor which provides data processing and analysis and operates the patch 100. The memory bank 112 stores data related to the patch or the impacts. The wireless transceiver 114 includes a transmitter and a receiver circuitry. Elements 108, 110, 112 and 114 could be designed in a single chip such as an ASIC (Application Specific Integrated Circuit), which is low power and smaller than a COTS solution. Likewise sensors 104 and 106 may also be designed into a single sensor. Ideally all components on the patch could be designed into one ASIC.

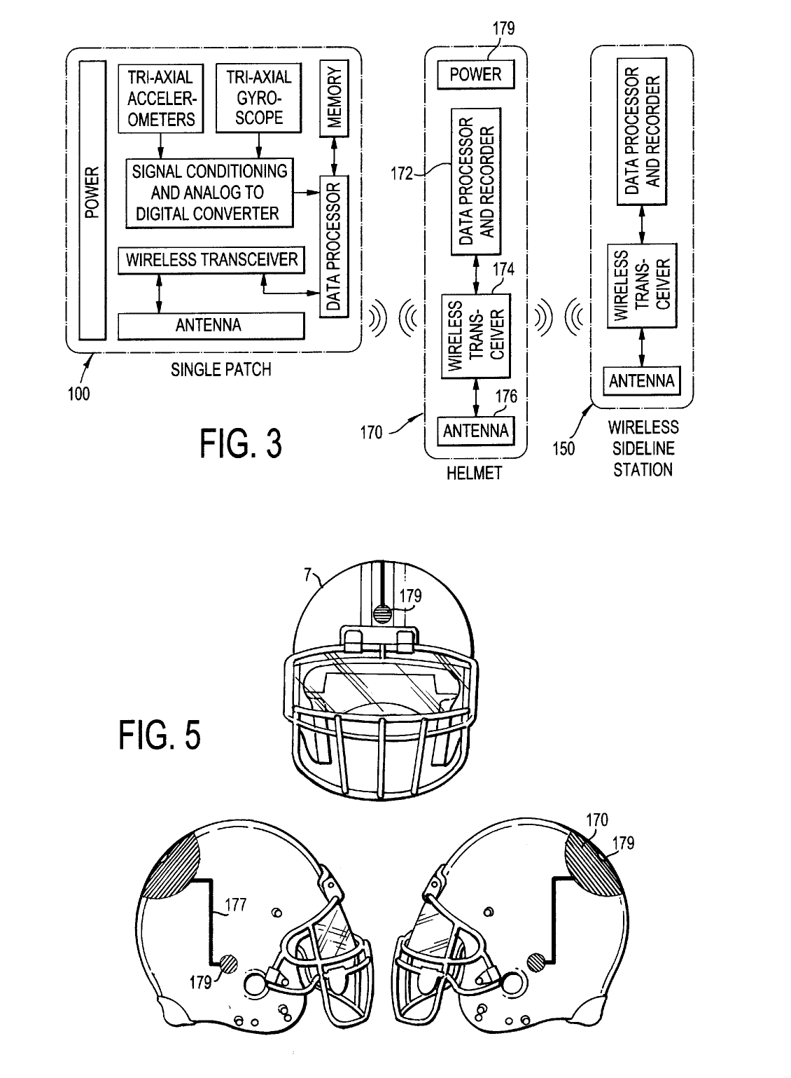

Turning to FIG. 3, a wireless repeater 170 can optionally be provided. The repeater 170 is mounted to or within a proximity garment worn by the user (e.g., a helmet or headgear). The wireless repeater could be a standard such as IEEE-802.11, known as WIFI, or Bluetooth, or a proprietary or custom protocol that can include encryption. The patch 100 operates the same as in FIG. 1, except now the digital sensed signal is transmitted to the repeater 170. The repeater 170 has a processor 172 with a storage device (such as a memory), a transceiver 174, an antenna 176, and a separate power supply 178 for supplying power to the repeater 170. The repeater processor 172 receives the digital sensed signal and/or alarm signal via the transceiver 174 and antenna 176. The transceiver 170 can store that information, boost the signal and retransmit it to the base station 150. The patch 100 and the repeater 170 can communicate on a first frequency, and the repeater 170 can communicate with the base station 150 on a second frequency, so that the communications do not cause interference with one another and so that the patch to repeater communication is lower in power than the repeater to sideline communication.

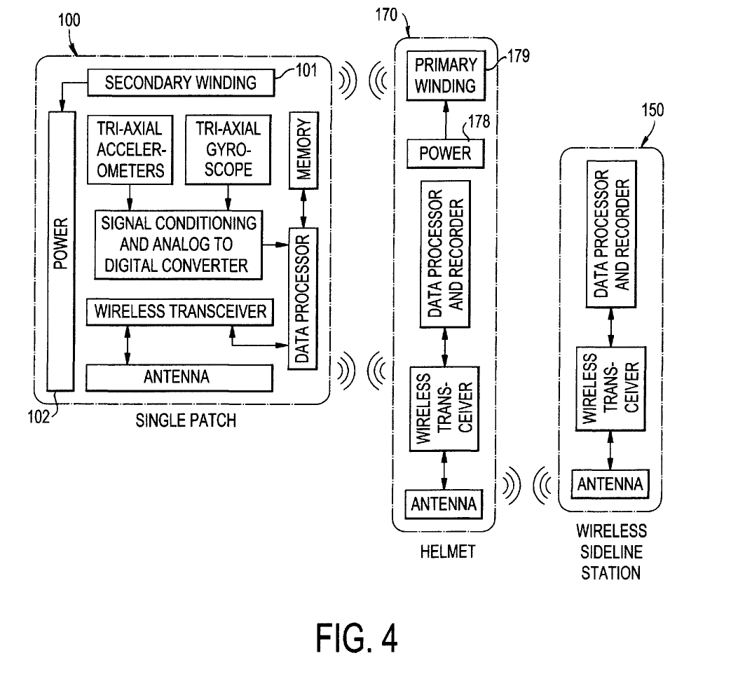

Referring now FIG. 4, the repeater 170 can also be used to provide power management to the patch 100. The patch 100 enters into a low- or no-power mode when the helmet of the repeater 170 is not worn by the player. When the helmet is worn, the repeater 170 provides wireless power to the patch 100. In this configuration, power is transferred inductively using a transformer. Accordingly, the repeater 170 is provided with a primary winding coil 179 which draws power from the power source 178, and the patch 100 is provided with a secondary winding coil 101. A varying current in the primary winding 179 located in the helmet creates a varying magnetic field in the secondary winding 101, which induces a varying voltage. Thus power is transferred from the helmet to the patch.

The primary winding 179 and the secondary winding 101 must be in close proximity to one another to provide an efficient transfer of power, so the primary winding 179 in the repeater 170 must be aligned with the secondary winding 101 of the patch 100. The efficiency of the power transmission between the secondary winding 101 and the primary winding 179 is related to the coil size, number of turns in the coils, and the distance separating the two coils 101, 179. The power transmission can therefore be modified as needed by varying these variables for a given helmet application. Power can also be transferred by other suitable means other than transformer coils.

Referring to FIG. 5, the implementation of the repeater 170 in a helmet 7 is shown. The power supply 178 is located in an area of the helmet 7 which is not likely to receive a large impact, such as the upper rear of the helmet, as shown. As also shown, the separate wireless power transfer module (primary winding 179) is at a position on the helmet 7 which is aligned with the secondary winding 101 of the sensor patch 100 (FIG. 2). The primary winding 179 can be at the same location as the power supply 178, or at a different position, in which case a power line 177 connects the power 178 to the winding 179. The primary windings 179 are located at the inside of the helmet 7 to be closer to the sensor patch 100, and can even come into direct contact with the patch 100. However, the primary winding 179 can also be located on the outside of the helmet 7, or at any other suitable location.

The power transfer from the helmet 7 to the patches will be greatly impacted in case of misalignment of the helmet 7 and might not operate. In case the repeater 170 is not able to communicate with the patches 100, the repeater 170 can send a message to the sideline base station 150 to warn the supporting staff of the issue. This can happen if the alignment is not good or if a patch 100 is defective, such as if the patch 100 doesn't respond to an interrogation signal from the repeater 170 or if the repeater 170 is not receiving a sensed signal from the patch 100. Furthermore, the repeater 170 can send positive alerts to the sideline base station 150 to let the supporting staff know that everything is ‘OK’ and the system is operating properly. If the base station 170 does not receive an ‘OK’ message from a given helmet, this will mean that the helmet is not operating properly. The ‘OK’ signal can occur at predetermined times, or in response to an interrogation signal sent from the base station 150.

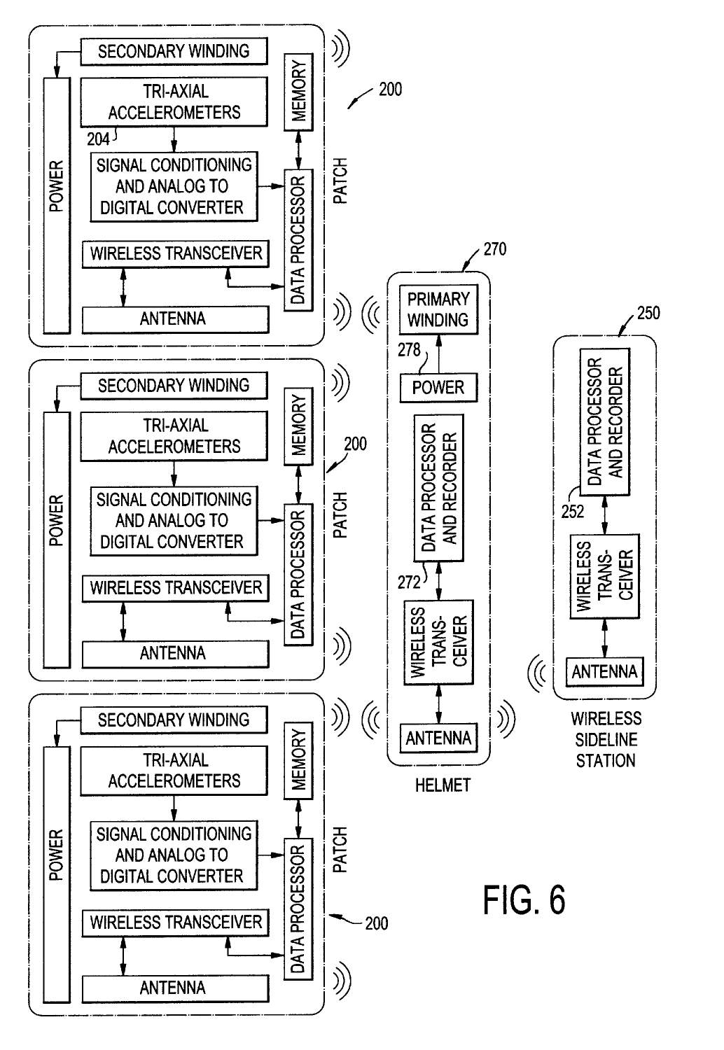

Turning to FIG. 6, three patches 200 are used for a single individual player, instead of the single patch 100 of FIGS. 1-4. The three patches 200 do not carry a gyroscope 106, but only have accelerometers 204. The three patches 200 utilize a single tri-axial accelerometer 104 to make them identical to one another. One benefit of having three patches 200 is that they only use accelerometers 204 and not gyroscopes 106.

The patches 200 can each directly transmit its respective sensed signal directly to the sideline base station 250 (i.e., without using the repeater 270), as in FIG. 1, or can optionally be paired with a repeater 270 located in the helmet, as in FIG. 3, which can also be used to power the patch 200, as in FIG. 4 (and as shown in FIG. 6). Since there are three patches 200, each one has its own unique ID and is associated with the particular individual. Where the repeater 270 also powers the patch 200, the repeater 270 has a primary winding 279 aligned with the secondary windings 201 in each of the patches 200. The operation is the same as discussed for FIG. 1. The patches 200 provide the same data as the single patch of FIG. 1. However, in the current embodiment, the base station 250 combines the data from the three patches 200 to determine the location of the impact.

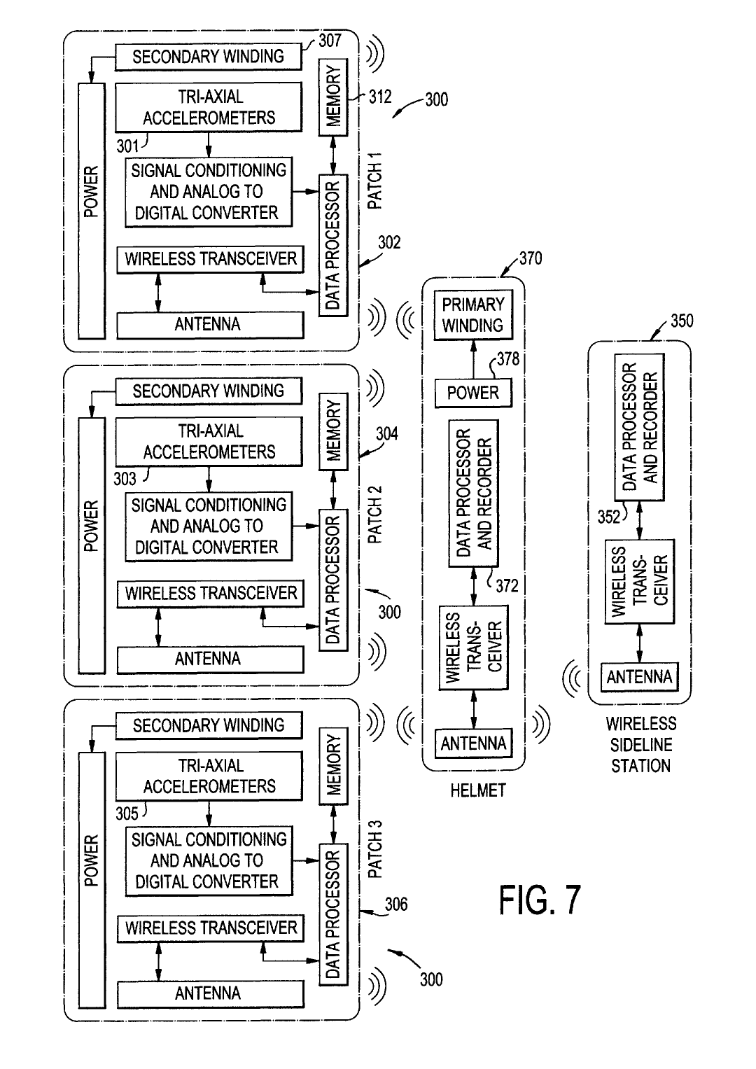

As shown in FIG. 7, another embodiment of the invention having three patches 300 is shown. One of the three patches 302 can have a tri-axial accelerometer 301, another patch 304 has a bi-axis accelerometer 303, and the third patch has a uni-axis accelerometer 305, respectively. In this case, the three patches 302, 304, 306 are different from one another since they each have different sensors 301, 303, 305, though the operation of the current embodiment is otherwise similar to that described in FIGS. 1 and 6. The patches 302, 304, 306 can transmit directly to the sideline base station 350 (i.e., without using the repeater 370), or can be paired with the repeater 370 located in the helmet, as in FIG. 3, which can also be used to power the patches 302, 304, 306, as shown. The repeater 370 has three primary windings 379, each of which are at one of the positions on the helmet 7 aligned to communicate with each of the sensor patches 300, as shown in FIG. 5.

The three patches 200, 300 provide a six degree of freedom sensor system which completely characterizes the movement of a user's head. These 6 degrees of freedom are comprised of three linear acceleration axes and three angular acceleration axes. As shown in FIG. 7, these axes can be obtained by one tri-axial sensor 301, one bi-axial sensor 303, and one uni-axial sensor 305 mounted directly on the head. As shown in FIG. 2, the first sensor patch 302 can be located on the user's forehead, the second sensor patch 304 is located behind the user's left ear, and the third sensor patch 306 is located behind the user's right ear. However, the same information can be obtained in FIG. 6 by mounting three tri-axial sensors 204 at each of the locations of FIG. 2, and ignoring or averaging the redundant axial information. However, other locations can be selected within the scope of the invention.

With respect to all of the embodiments, the sensor(s) 100, 200, 300, relay 170, 270, 370, and base station 150, 250, 350 each have bidirectional communication capability. The remote base station processor 152, 252, 352 can directly interrogate the sensor patch(es) 100, 200, 300 and/or the helmet repeater 170, 270, 370 to access any information stored in the processors 110, 172, 210, 272, 310, 372 or the associated memory 112, 212, 312 respectively. The repeater processor 172, 272, 372 or its associated memory can also store a unique user identification (ID) which identifies the patches 100, 200, 300 with which it communicates, and in the case of having multiple patches 100, 200, 300 each can also have a separate ID or no ID. The ID can be programmed into the sensor patch(es) 100, 200, 300 or the repeater 170, 270, 370 just prior to the sensor patch(es) 100, 200, 300 and the repeater 170, 270, 370 being placed on the user. Or, the ID can be preprogrammed into the processor memory 112, 212, 312 and read from the helmet processor 172, 272, 372. The helmet processor 172, 272, 372 can also have a memory or storage device which also stores a unique ID for the helmet, which can be the same or different from the unique ID of the sensor patch(es) 100, 200, 300. The data between the repeater 170, 270, 370 and the patch(es) 100, 200, 300 can be transferred by several different means, including infra red, radio frequency, ultrasonic, wired, and modulation over power.

Still referring to FIGS. 1-7, the patches 100, 200, 300 have the capability to transfer impact data directly to the base station 150, 250, 350 at a remote location such as sideline of a football field. The data is transferred using wireless communication, such as RF. The data can be continuously transferred in real-time from the patches 100, 200, 300 directly to the base station 150, 250, 350 or via a repeater 170, 270, 370 to provide immediate information about the condition of the wearer. Or, the data can be stored in the helmet processor 172, 272, 372 memory or the sensor memory 112, 212, 312 and downloaded to the base station 150, 250, 350 after the game. The initiation of the data transmission can come from the remote station 150, 250, 350 or the repeater 170, 270, 370. The patches 100, 200, 300 have the capability to transfer its data or an alarm to the sideline receiver 150, 250, 350 in case of an emergency based on an initial set of criteria. In case a player was to experience a head impact above a ‘danger’ threshold, the patch 100, 200, 300 can send an alarm. Such threshold can be changed, and predetermined by medical staff at the base station 150, 250, 350 and reprogrammed in each of the individual patches 100, 200, 300. Thus, one player could have a different critical or ‘danger’ threshold than another player based on the player's head impact history and other physical attributes. The repeater 170, 270, 370 can also be preprogrammed with thresholds for the individual player that may include the level of shock received, the number of impacts received in a certain time period, etc. Other sensory data can also be included on the sensor patch 100, 200, 300 such as a heart rate monitor or a temperature sensor, and this information can also be transmitted from the headgear to the base station 150, 250, 350. In addition to a wireless transmission, the repeater 170, 270, 370 can be interrogated/transfer data by a local hardware/wired connection as well.

The information can also be transmitted from the patch 100, 200, 300, the base station 150, 250, 350 and/or from the repeater 170, 270, 370 to a hand held device, such as a pager or a cell phone, which can be carried and/or worn by the medical staff. An exemplary handheld device is disclosed in WO/2009/006406. Once the staff gets an alert, they can then request that the player or individual in question be removed from the game or from the field of play for immediate examination. This approach assures that the player or individual does not risk further injury by deciding to stay in the field of play after an event which may render the player incapable of making that decision.

In the embodiments where a repeater 170, 270, 370 is provided, in order to limit or reduce tampering, each player has his own unique helmet 7. Players cannot exchange helmets since they are individually fitted to their head's size and shape which guarantees that there is no tampering of the recording devices. Powering the sensor patch(es) 100, 200, 300 also limits players from tampering with the sensors since they are not powered until the player puts a helmet on. However, in the event that the helmets 7 themselves are not unique, or in the event that a player puts the wrong helmet on, the sensor patch(es) 100, 200, 300 and helmet electronics can be paired by the sideline staff using the sideline computer or handheld devices. The pairing process will give the patch(es) the players unique Identification Number or ID. Accordingly, correspondence between a specific player and specific sensor patches can be maintained and monitored.

The present invention does not measure erroneous head trauma events. Since the patch(es) 100, 200, 300 are directly attached to the wearer's body, and not the headgear, any event recognized by the patch 100, 200, 300 is due to a direct impact to the wearer's body. In addition, the embodiments where the repeater 170, 270, 370 powers the patch(es) 100, 200, 300 (such as FIGS. 4, 6, 7), the patch(es) 100, 200, 300 lose power when the headgear 7 is removed since the secondary winding 101, 201, 301 is effectively disconnected from the primary winding 179, 279, 379 and the power source 178, 278, 378. In the embodiments where the patch(es) 100, 200, 300 have batteries 102, 202, 302 (such as shown in FIG. 1), the repeater 170, 270, 370 can communicate with the patch(es) 100, 200, 300 so that the removal of the helmet 7 can be determined. For instance, the repeater 170, 270, 370 can send a message at a preprogrammed timing to let the patch(es) 100, 200, 300 know that the helmet 7 is still on the player's head, using ultra low range wireless transmission. Once the helmet 7 is removed, the patch(es) 100, 200, 300 will no longer receive the low range wireless transmission.

Thus, the system 5 will not recognize impacts to the helmet when the helmet 7 is not being worn. For instance, the system 5 will not recognize an impact when a player takes his helmet 7 off, and drops it on the ground or throws it. The present system limits tampering as the sensor and the recorder have to be placed in proximity to operate. The system 5 does not detect irrelevant events such as an improper use.

The invention also contemplates the case in which the helmet 7 is separated from the player's head during a legitimate impact during use. Once the helmet 7 is removed (whether from a legitimate impact or the player removing the helmet), the patch processor 110, 210, 310 directs the memory (such as a small buffer) 112, 212, 312 to continue to record and save the last impact. It doesn't matter if the player takes the helmet off or if it is knocked off, the patch(es) 100, 200, 300 will continued to record for several seconds, and will immediately transmit the data to the base station 150, 250, 350 and/or transmit the data to the repeater 170 when the helmet 7 is replaced. Since the sensor patch(es) 100, 200, 300 is directly fixed to the user's head, the system 5 will record the impact to the user's head, but not record any impact to the helmet which has come off. The memory can then be interrogated by the base station 150, 250, 350, or can be transferred to the headgear repeater 170, 270, 370 if the headgear is put back on the wearer.

If the patch(es) 100, 200, 300 are powered by the repeater 170, 270, 370, the patch(es) 100, 200, 300 can be provided with a power storage which is recharged while the patch 100, 200, 300 is in normal use. The power storage can provide the patch(es) 100, 200, 300 with temporary power when the helmet 7 is removed. The length of the autonomous mode is based on the patch's size and other impact signature factors. In addition, an ON/OFF button can be utilized on the sensor electronics 100, 200, 300 and/or headgear electronics 170, 270, 370 to turn the system off when not in use.

Although a specific embodiment is directed to head impact measurement, it will be understood that the present invention is not so limited. The invention is directed to a sensor to be mounted on the body of a user to monitor an impact or other measurement (such as heart rate, temperature, etc.). The sensor can be (as the helmet is not required) matched with a proximity device or apparatus which provides power and can record, store and transmit data without such apparatus being physically connected to the sensor. It will be understood that sensors located at other body locations and to monitor events other than impacts are contemplated. In addition, while a single patch 100 and three patches 200, 300 are discussed in various embodiments of the invention; it should be appreciated that any suitable number of patches can be provided.

It is noted that the present invention has either one sensor patch 100 at a single location on the player's head (such as in FIG. 1), or three sensor patches 200, 300 each at one of three different locations on the player's head (such as in FIGS. 6, 7). To make the measurements on the head, six degrees of freedom are needed: three linear acceleration measurements x, y and z and three angular acceleration measurements to obtain the rotation about the x, y, and z axes. In the embodiments having a single patch, one tri-axial accelerometer is utilized for the linear x, y, and z and one tri-axial gyroscope is utilized for the rotation about the x, y and z axes. Accordingly, the one patch needs these two tri-axial sensors.

However, this is not always feasible because the gyroscopes can be large. So, another way to get the six degrees of freedom is to utilize three patches that have a combined total of six linear accelerometer channels. The three patches 300 (FIG. 7) include at least one that has a tri-axial accelerometer 301, one that has a bi-axial accelerometer 303, and one that has a single axis accelerometer 305. But, the present invention also provides for a single uniform patch which can be interchangeably used and replaced without having to determine whether it is a tri-axial accelerometer, biaxial accelerometer or single axis accelerometer. Thus, in FIG. 6, the three patches 200 are each a tri-axial accelerometer 204 and therefore provide redundant data for 3 axes that can be used to average and reduce error. In this case each tri-axial patch 200 sends its orientation information, derived from the DC component of acceleration, along with its impact information to the sideline station 250 directly or via the repeater 270 to the sideline station 250. Based on this information the sideline station 250 can determine the redundant axes and average the data from the redundant axes to reduce an error that is associated with the measurements. If the data from the redundant axes are extremely different from the primary axes, then the sideline unit 250 can also display an output message which can alert the medical staff to check the patches 200 on the player to see if any patches are mounted inappropriately, damaged, or not working properly.

So, the different patch embodiments are as follows: (a) one patch 100 (FIGS. 1, 3, 4) with a tri-axial accelerometer 104 and a tri-axial gyroscope 106, (b) three patches 300 (FIG. 7) one with a tri-axial accelerometer 301, one with a biaxial accelerometer 303, and one with a single axis accelerometer 305, and finally (c) three patches 200 (FIG. 6) each with a tri-axial accelerometer 204. For the location of the three patches 200, 300 (either embodiment), the distance between the two patches behind each ear (which defines the x axis) and then the distance from the forehead sensor to the X axis, is needed to be known. A generic predetermined number can be used for each of these based on the average dimensions of a human head (though some error is introduced). Or, the measurement for each player can be stored in the database so that it can be accessed when calculating the final impact direction and amplitude for each hit.

The foregoing description and drawings should be considered as illustrative only of the principles of the invention. The invention may be configured in a variety of shapes and sizes and is not intended to be limited by the preferred embodiment. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

What is claimed is:

1. A system for monitoring body signals comprising:
a single sensor patch to be directly mounted to a body of an individual user to be monitored, said single sensor patch having a sensor element capable of sensing a condition of the user's body and providing a sensed signal based on the sensed condition of the user's body, and a sensor wireless transmitter capable of receiving the sensed signal and transmitting the sensed signal; and,
a base station located remote from said single sensor patch, said base station having a base station wireless receiver capable of receiving the sensed signal directly from said sensor wireless transmitter in real-time, and a base station processor capable of analyzing the received sensed signal and determining the condition of the user's body based on the analyzed sensed signal.

2. The system of claim 1, wherein said single sensor patch is located on a sporting event field of play and said base station is located on a side of the field of play.

3. The system of claim 1, wherein said single sensor patch comprises a first single sensor patch and the individual user is a first individual user, and further comprising a second single sensor patch to be mounted to a body of a second individual user.

4. The system of claim 1, wherein said sensor element comprises a tri-axial accelerometer and a tri-axial gyroscope.

5. The system of claim 1, wherein said single sensor patch is associated with a unique identification (ID) and said sensor wireless transmitter transmits the sensed signal with the unique ID.

6. The system of claim 1, further comprising a sensor processor capable of receiving the sensed signal, detecting an alarm condition and generating an alarm signal, and wherein said sensor wireless transmitter transmits the alarm signal and said base station processor receives the alarm signal.

7. The system of claim 1, wherein no repeater is utilized between said single sensor patch and said base station.

8. The system of claim 1, said single sensor patch further comprising a sensor battery to power said sensor element and said sensor wireless transmitter.

9. The system of claim 1, further comprising three sensor patches to be directly mounted to the body of the individual user, said three sensor patches comprising a first sensor patch mounted to a forehead of the user, a second sensor patch mounted at a right side of a head of the user, and a third sensor patch mounted to a left side of the user's head, wherein each of said first, second and third sensor patches have a sensor element comprising a single tri-axial sensor.

10. A system for monitoring body signals comprising:
a single sensor patch to be directly mounted to a body of an individual user to be monitored, said single sensor patch having a sensor element capable of sensing a condition of the user's body and providing a sensed signal based on the sensed condition of the user's body, and a sensor wireless transmitter capable of receiving the sensed signal and transmitting the sensed signal;
a proximity garment having a proximity receiver capable of receiving the sensed signal directly from said single sensor patch and a proximity transmitter capable of transmitting the sensed signal in real-time; and,
a base station located remote from said single sensor patch, said base station having a base station wireless receiver capable of receiving the sensed signal directly from said proximity transmitter in real-time, and a base station processor capable of and analyzing the received sensed signal and determining the condition of the user's body based on the analyzed sensed signal in real-time.

11. The system of claim 10, wherein said single sensor patch has a sensor battery to power said sensor element and said sensor wireless transmitter, and said proximity garment has a proximity battery to power said proximity receiver and said proximity transmitter.

12. The system of claim 10, wherein said proximity garment has a proximity battery and a proximity power transfer module, and said single sensor patch has a sensor power transfer module, and said proximity power transfer module transfers power from said proximity battery to said sensor power transfer module to power said sensor element and said sensor wireless transmitter.

13. A system for monitoring body signals comprising:
a proximity garment to be worn by a user to be monitored;
one or more sensors to be directly mounted to a body part of the user to be monitored;
electronics mounted within said proximity garment and aligned with said one or more sensors when said one or more sensors are mounted to the body of the user to be monitored;
power circuitry mounted within said proximity garment for powering said electronics and said one or more sensors;
a storage device capable of storing data in said proximity garment; and
a transmitter capable of transferring data from said proximity garment to a location separate from a location of said user.

14. The system of claim 13, wherein said sensors and said electronics are operable when said one or more sensors and electronics are in close proximity when said proximity garment is worn by the user having said one or more sensors mounted to the user's body.

15. The system of claim 13, wherein said one or more sensors are mounted within no more than one sensor patch which is capable of being directly mounted to the body part of the user.

16. The system of claim 13, wherein said proximity garment comprises a helmet.

17. The system of claim 13, further comprising a base station located remote from said proximity garment, said base station having a base station receiver capable of directly receiving the data transferred from said transmitter.

18. A system for monitoring a user's body, comprising:
a proximity garment to be worn by the user;
a sensor capable of being directly mounted to the user's body and having sensor electronics comprising:
a sensor element capable of sensing a condition of the user's body and providing a sensed signal based on the sensed condition of the user's body;
a sensor wireless transmitter capable of receiving the sensed signal and transmitting the sensed signal;
a sensor power module capable of wirelessly receiving power and providing power to said sensor element and said sensor wireless transmitter;
a proximity garment power module located in said proximity garment and in close proximity with said sensor power module, wherein said proximity garment power module is capable of wirelessly transferring power to said sensor power module only when said sensor proximity module is in close proximity to said proximity garment power module;
a proximity garment wireless receiver located in said proximity garment and in close proximity with said sensor wireless transmitter, wherein said proximity garment wireless receiver is capable of receiving the sensed signal from said sensor wireless transmitter only when said proximity garment wireless receiver is in close proximity to said sensor wireless transmitter; and,
a proximity garment wireless transmitter for wirelessly transmitting the sensed signal received by said proximity garment wireless receiver.

19. The system of claim 18, wherein the sensor electronics do not analyze the sensed signal.

20. The system of claim 18, said sensor electronics further including a storage device for storing the sensed signal.

21. The system of claim 18, wherein the sensor power module does not receive power from said proximity garment power module when said proximity garment power module is not in close proximity to said sensor power module.

22. The system of claim 21, wherein said sensor element stops sensing when said proximity garment power module is not in close proximity to said sensor power module.

23. The system of claim 18, wherein said sensor electronics further comprises a temporary power storage capable of being charged by said sensor power module when said proximity garment power module is in close proximity to said sensor power module, and temporarily provides power to said sensor element and said sensor transmitter when said proximity garment power module is not in close proximity to said sensor power module.

24. The system of claim 18, wherein said proximity garment power module comprises a primary coil of a transformer, and said sensor power module comprises a secondary coil of the transformer.

25. The system of claim 18, said sensor electronics further comprising an analog-to-digital converter capable of receiving the sensed signal from said sensor element, converting the sensed signal to a digital sensed signal, and providing the digital sensed signal to said sensor wireless transmitter for transmission.

26. The system of claim 18, wherein said sensor is mounted within no more than one sensor patch which is to be mounted to the body part of the user.

27. The system of claim 26, further comprising three sensor patches to be mounted to different locations on a head of a single individual user, wherein each of said three sensor patches comprise a single tri-axial sensor.

28. The system of claim 18, further comprising a base station having a base station processor, a sensor memory storing a unique sensor identification (ID) for the sensor, a proximity garment memory located in said proximity garment for storing a unique proximity garment ID for the proximity garment, said sensor wireless transmitter transmitting the sensor ID, wherein said proximity garment wireless receiver receives the sensor ID and said proximity garment wireless transmitter transmits the sensor ID and the proximity garment ID to the base station processor with the sensed signal and the base station processor associates the sensor ID and the proximity garment ID with the user.
